(19) United States
(12) Patent Application Publication
(10) Pub. No.: US 2008/0182848 A1
(43) Pub. Date: Jul. 31, 2008

(54) Novel Process

(76) Inventors: Debashish Datta, Maharashtra (IN); Ajaysingh Rawat, Maharashtra (IN); Shardard N. Duche, Maharashtra (IN); E.K.S. Vijayakumar, Maharashtra (IN)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/961,673

(22) Filed: Dec. 20, 2007

Related U.S. Application Data
(63) Continuation of application No. PCT/GB06/50166, filed on Jun. 21, 2006.

(30) Foreign Application Priority Data
Jun. 21, 2005 (IN) 728/MUM/2005

(51) Int. Cl. A61K 31/4985; C07D 487/04; A61P 25/22; A61P 25/24; A61P 25/00

(52) U.S. Cl. 514/249; 544/350

(57) ABSTRACT

The present invention relates to a process for optically resolving eszopiclone, comprising chiral chromatography. Preferably the process comprises a multi-column continuous process or a simulated moving bed process. Preferably the stationary phase used in the chiral chromatography process comprises an amylose or cellulose derivative of tris(3,5-dimethylphenyl carbamate), or an amylose derivative of tris-α-methylbenzylcarbamate. The process of the present invention has the advantage that it is high yielding and can be carried out on an industrial scale.

The present invention also provides eszopiclone, or a pharmaceutically acceptable salt thereof, obtained by the chiral chromatography process. The eszopiclone or salt thereof is suitable for use as a medicament, for example, for the treatment of anxiety or insomnia.

### CROSS REFERENCE TO RELATED APPLICATION(S)

This application is a Continuation of International Patent Application PCT/GB2006/050166, filed on Jun. 21, 2006, which claims priority to India Application No. 728/mum/2005, filed Jun. 21, 2005, the entire contents of both of which are hereby incorporated by reference.

### TECHNICAL FIELD

The present invention relates to a process for the separation of the dextrorotatory isomer of 6-(5-chloro-2-pyridyl)-5[(4-methyl-1-piperazinyl)-carbonyloxy]-7-oxo-6,7-dihydro-5H-pyrrolo-[3,4b]-pyrazine (zopiclone) or eszopiclone from the racemic mixture.

### BACKGROUND ART

Zopiclone, chemically named (±) of 6-(5-chloro-2-pyridinyl)-6,7-dihydro-7-oxo-5H-pyrrolo-[3,4b]-pyrazin-5-yl-4-methylpiperazine-1-caboxylate, of Formula (I) is a non-benzodiazepine hypnotic.

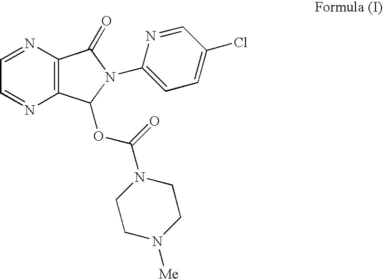

Zopiclone and its optically pure enantiomers are useful in the treatment of diseases and conditions including epilepsy, anxiety, aggressive behavior, muscle tension, behavioral disorders, depression, schizophrenia and endocrine disorders. Racemic zopiclone has been used to improve sleep in adults and geriatric patients with several types of sleep disorders including situational, transient and chronic insomnia of primary and secondary nature.

Zopiclone binds at or near benzodiazepine receptor complexes. These complexes are located both within the central nervous system and peripherally, and contain macromolecular complexes, which comprise benzodiazepine and GABA binding sites.

Eszopiclone is the S-isomer of the racemic product zopiclone. As the S-isomer of zopiclone, eszopiclone has been designed to be more specific for the GABA binding sites and possesses an approximately 50-fold higher binding affinity to GABA-A-receptors than the R-enantiomer. Also, the hypnotic activity of S-zopiclone has been reported to be two-fold more than racemic zopiclone.

The separation of two enantiomers present in a racemic mixture or any mixture of enantiomers is called resolution. Enantiomers are not readily separated by conventional means, such as recrystallisation or fractional distillation, since they have the same solubilities, melting points, boiling points, etc. Therefore special means are required for the resolution of two enantiomers. Prior art methods for the separation of eszopiclone are mentioned below.

### Chemical Resolution

U.S. Pat. No. 6,339,086 and U.S. Pat. No. 6,319,926 disclose the chemical resolution of precursors to yield eszopiclone.

Enantiomers have the same solubility profile and cannot be readily separated by simple recrystallisation, but diastereoisomers generally have different solubilities in some selected solvents. Disadvantages of chemical resolution:
The overall process of separation is time consuming, as it involves many steps.
If the drug is very sensitive to acid/base, degradation of the isomers occurs at the step of separation.
Recovery of both isomers is very difficult, as the addition of acid/base degrades isomers.
The overall yield of the final product is very low, increasing costs to a large extent.

In the case of zopiclone, the drug is extremely sensitive to pH variations and not an ideal member for separation by chemical means. For example, in U.S. Pat. No. 6,339,086, (±)-zopiclone is converted into diastereomeric salts using D-malic acid and eszopiclone is obtained in an overall yield of 36%. In U.S. Pat. No. 6,319,926, (±)-zopiclone is converted into diastereomeric salts using D(+)-O,O′-dibenzoyltartaric acid and eszopiclone is obtained in an overall yield of only 23%. Thus these methods are unsuitable for industrial scale manufacture of eszopiclone.

### Enzymatic Resolution

ES 2,101,653 discloses an enzymatic resolution of a racemic mixture of compounds to yield optically pure eszopiclone.

Enzymes are proteins which have many chiral centers and which occur in nature as a single enantiomer. The rates of reaction of two enantiomers with a single enantiomer of any chiral substance are different. The products will be diastereoisomeric and thus of different energies, and therefore the rates of formation of these products will in general be different. Enzymes are particularly effective in making this distinction, so that a racemic mixture can often be easily resolved by reaction with some simple substance in the presence of the chiral enzyme as catalyst. The enantiomer which reacts faster will be converted to a new compound having an entirely different functional group, while the enantiomer which reacts more slowly will remain unreacted.

### Disadvantages of Enzymatic Resolution:

Enzymes are very expensive which makes the overall process of separation expensive.
Enzymatic processes may not work on the final racemate but on selective intermediates and therefore additional conversion of the racemate to be resolved needs to be done to the intermediates on which the enzymes can selectively act.
Enzymatic reactions generally occur at high dilutions and the entire process requires a lot of water, which becomes difficult on large scale.
Degradation of the isomers is possible.
It is difficult to recover optically pure isomer.
The overall yield is low.

In case of zopiclone the enzymatic resolution has been developed not on a phenyl carbamate ester intermediate, but on other carbamate esters such as allyl, which has to be hydrolyzed to the alcohol intermediate and which in turn then has to be coupled with N-methyl piperazine via its phenyl carbamate ester to produce S-zopiclone. These additional steps cause problems in reducing the yield of the final eszopiclone. Thus enzymatic methods are unsuitable for industrial scale manufacture of eszopiclone.

### Asymmetric Synthetic Process

Single enantiomer can be prepared via an asymmetric synthetic process, which affords the single enantiomer directly with no further need for resolution of a racemic mixture. However, asymmetric synthesis gives low yields and the reagents used are expensive and not environmentally friendly.

### Chiral Separation

Chiral separation of optically active enantiomer from racemic mixtures is achieved by chromatographic separation using HPLC and GC, e.g. resolution of the racemic mixture directly using chiral stationery phase or by derivatising the racemic mixture into a diastereomeric mixture and separation of the diastereomers using a standard stationery phase. The later option further requires chemical conversion of one separated diastereomer into the required enantiomer. However, in practice, these chromatographic resolution techniques generally fail to afford commercial quantities of the desired pure enantiomer and are generally only used for production of small laboratory scale amounts.

U.S. Pat. No. 5,641,404 discloses a process for separating enantiomeric mixtures, comprising the steps of:
(a) contacting a liquid phase with a chiral phase, the liquid phase comprising methanol and pentane, the concentration of methanol being greater than one tenth percent on a volume basis and less than or equal to the saturation concentration of methanol in the liquid phase, and
(b) contacting an enantiomeric mixture with the chiral phase and with the liquid phase, the concentration of pentane in the liquid phase being at least that necessary to resolve the enantiomeric mixture into its enantiomers with a resolution at least one and one half times greater than when the pentane of the liquid phase is replaced with hexane.

U.S. Pat. No. 5,641,404 also discloses a chiral phase comprising a polysaccharide derivative selected from the group consisting of cellulose and amylose derivatives. However, it does not mention selectively the use of specific columns for specific drugs.

U.S. Pat. No. 5,889,180 discloses the separation of chiral materials by simulated moving bed chromatography using a chiral stationary phase having silica as an inert core support and an achiral eluent, the improvement comprising using a chiral stationary phase in which the silica has a pore size between about 50 and about 100 angstroms.

Consequently, the known processes for the preparation of eszopiclone are not suitable for industrial scale manufacture. Therefore there is a need for a more efficient process for optically resolving eszopiclone on a manufacturing scale.

### OBJECTS OF THE INVENTION

It is an object of the present invention to provide a multi-column continuous process for resolving optically pure zopiclone using an amylose derivative of tris(3,5-dimethylphenyl carbamate), a cellulose derivative of tris(3,5-dimethylphenyl carbamate), or an amylose derivative of tris-α-methylbenzylcarbamate as a stationary phase.

It is a further object of the present invention to reduce the total number of steps required for the resolution of zopiclone.

It is a further object of the present invention to provide a process for optically resolving zopiclone, which yields optically pure eszopiclone.

It is yet another object of the present invention to provide a process for the resolution of zopiclone, which gives high yield.

It is yet a further object of the present invention to provide an economical process for the resolution of zopiclone.

### SUMMARY OF THE INVENTION

According to a first aspect of the present application, there is provided a process for optically resolving eszopiclone, comprising chiral chromatography.

For the purposes of the present invention, the term ‘eszopiclone’ is defined as (+)-zopiclone. Zopiclone is (±)-6-(5-chloro-2-pyridinyl)-6,7-dihydro-7-oxo-5H-pyrrolo-[3,4b]-pyrazin-5-yl-4-methylpiperazine-1-caboxylate of Formula (I).

For the purposes of the present invention, the term ‘optically resolving’ is defined as separating eszopiclone from a mixture of eszopiclone and (−)-zopiclone, such that the eszopiclone obtained has an enantiomeric excess (ee) of 90% or more, preferably 95% or more, and even more preferably 98% or more. Preferably the eszopiclone obtained is substantially free of (−)-zopiclone.

For the purposes of the present invention, the term ‘chiral chromatography’ is defined as chromatography using a chiral stationary phase and a mobile phase.

Preferably the process comprises a multi-column continuous process or a simulated moving bed process.

Preferably the stationary phase used in the chiral chromatography process comprises an amylose derivative of tris(3,5-dimethylphenyl carbamate), a cellulose derivative of tris(3,5-dimethylphenyl carbamate), or an amylose derivative of tris-α-methylbenzylcarbamate.

The stationary phase used in the chiral chromatography process may comprise a silica gel coated with a functionalized polysaccharide. Preferably the stationary phase comprises Chiralcel® OD, Chiralpak® AD or Chiralpak® AS.

Preferably the mobile phase used in the chiral chromatography process comprises an alcohol, another organic solvent, or a mixture thereof. The alcohol may comprise methanol, ethanol, propanol, isopropanol, or a mixture thereof. The organic solvent may comprise acetonitrile or hexane. Optionally, the mobile phase may further comprise an organic amine co-solvent, such as dimethylamine, trimethylamine or isopropylamine. Optionally, the mobile phase comprises a mixture of an alcohol and another organic solvent, such as a mixture of methanol and acetonitrile or a mixture of isopropanol and hexane.

For economic efficiency, the mobile phase used in the chiral chromatography process is preferably recycled.

In a preferred embodiment of the present invention, there is provided a process for optically resolving eszopiclone comprising:

a chiral stationary phase of an amylose derivative of tris(3,5-dimethylphenyl carbamate), a cellulose derivative of tris(3,5-dimethylphenyl carbamate), or an amylose derivative of tris-α-methylbenzylcarbamate, and

a mobile phase of an alcohol, an organic solvent, or a mixture thereof.

Preferably the chiral chromatography process is carried out at a temperature of 15-40° C.

Racemic or enantiomerically enriched zopiclone can be resolved by the chiral chromatography process of the present invention. Preferably racemic zopiclone is resolved.

For the purposes of the present invention, the term ‘racemic zopiclone’ is defined as a mixture of eszopiclone: (−)-zopiclone in the ratio of 55:45 to 45:55, preferably in the ratio of about 50:50. The term ‘enantiomerically enriched zopiclone’ is defined as a mixture, wherein the percentage of eszopiclone is greater than the percentage of (−)-zopiclone. Typically ‘enantiomerically enriched zopiclone’ is a mixture of eszopiclone: (−)-zopiclone in the ratio of 100:0 to 55:45, preferably in the ratio of 90:10 to 60:40, more preferably 90:10 to 70:30.

The process of the present invention has the advantage that it can be carried out on an industrial scale. For the purposes of the present invention, the term ‘industrial scale’ is defined as a per day production of 0.5 kg or more of eszopiclone, preferably 1 kg or more, more preferably 10 kg or more, more preferably 20 kg or more, and even more preferably 50 kg or more.

The process of the present invention has the further advantage that it is high yielding. The yield of the eszopiclone produced can be as high as 70%, 80%, 90%, 95% or more of the theoretical yield. For the purposes of the present invention, the term ‘theoretical yield’ is defined as the theoretical maximum yield of an enantiomer based on the quantity of the enantiomer in the starting mixture prior to the chiral chromatography process of the present invention.

According to a second aspect of the present application, there is provided eszopiclone, or a pharmaceutically acceptable salt thereof, obtained by a chiral chromatography process of the present invention.

For the purposes of the present invention, a ‘salt’ of eszopiclone is an acid addition salt. Acid addition salts are preferably pharmaceutically acceptable, non-toxic addition salts with suitable acids, including but not limited to inorganic acids such as hydrohalogenic acids (for example, hydrofluoric, hydrochloric, hydrobromic or hydroiodic acid) or other inorganic acids (for example, nitric, perchloric, sulphuric or phosphoric acid); or organic acids such as organic carboxylic acids (for example, propionic, butyric, glycolic, lactic, mandelic, citric, acetic, benzoic, salicylic, succinic, malic or hydroxysuccinic, tartaric, fumaric, maleic, hydroxymaleic, mucic or galactaric, gluconic, pantothenic or pamoic acid), organic sulphonic acids (for example, methanesulphonic, trifluoromethanesulphonic, ethanesulphonic, 2-hydroxyethanesulphonic, benzenesulphonic, toluene-p-sulphonic, naphthalene-2-sulphonic or camphorsulphonic acid) or amino acids (for example, ornithinic, glutamic or aspartic acid).

Preferably the eszopiclone or salt thereof is suitable for use as a medicament. The medicament may be suitable for the treatment of anxiety such as acute anxiety, chronic anxiety or a general anxiety disorder; a convulsive state or disorder such as epilepsy or epileptic seizures; an affective disorder such as depression, attention deficit disorder (ADD) or attention deficit disorder with hyperactivity (ADDH); a sleep disorder such as insomnia including situational, transient and chronic insomnia of a primary and secondary nature; aggressive behavior; spasticity or acute muscle spasm; muscle tension; a behavioral disorder; a schizophrenic disorder; a disease or condition associated with abnormal plasma hormone levels such as an endocrine disorder; alcohol or drug addiction, symptoms of drug withdrawal or symptoms of alcohol withdrawal. The medicament may be suitable for improving sleep quality or time.

According to a third aspect of the present application, there is provided a pharmaceutical composition comprising eszopiclone or a salt thereof, and a pharmaceutically acceptable carrier or diluent. Preferably the pharmaceutical composition is suitable for the treatment of anxiety such as acute anxiety, chronic anxiety or a general anxiety disorder; a convulsive state or disorder such as epilepsy or epileptic seizures; an affective disorder such as depression, attention deficit disorder (ADD) or attention deficit disorder with hyperactivity (ADDH); a sleep disorder such as insomnia including situational, transient and chronic insomnia of a primary and secondary nature; aggressive behavior; spasticity or acute muscle spasm; muscle tension; a behavioral disorder; a schizophrenic disorder; a disease or condition associated with abnormal plasma hormone levels such as an endocrine disorder; alcohol or drug addiction, symptoms of drug withdrawal or symptoms of alcohol withdrawal. Preferably the pharmaceutical composition is suitable for improving sleep quality or time.

According to a fourth aspect of the present application, there is provided a use of eszopiclone or a salt thereof, for the manufacture of a medicament for the treatment of anxiety such as acute anxiety, chronic anxiety or a general anxiety disorder; a convulsive state or disorder such as epilepsy or epileptic seizures; an affective disorder such as depression, attention deficit disorder (ADD) or attention deficit disorder with hyperactivity (ADDH); a sleep disorder such as insomnia including situational, transient and chronic insomnia of a primary and secondary nature; aggressive behavior; spasticity or acute muscle spasm; muscle tension; a behavioral disorder; a schizophrenic disorder; a disease or condition associated with abnormal plasma hormone levels such as an endocrine disorder; alcohol or drug addiction, symptoms of drug withdrawal or symptoms of alcohol withdrawal. According to the fourth aspect of the present application, there is also provided a use of eszopiclone or a salt thereof, for the manufacture of a medicament for improving sleep quality or time.

According to a fifth aspect of the present application, there is provided a method of treating anxiety such as acute anxiety, chronic anxiety or a general anxiety disorder; a convulsive state or disorder such as epilepsy or epileptic seizures; an affective disorder such as depression, attention deficit disorder (ADD) or attention deficit disorder with hyperactivity (ADDH); a sleep disorder such as insomnia including situational, transient and chronic insomnia of a primary and secondary nature; aggressive behavior; spasticity or acute muscle spasm; muscle tension; a behavioral disorder; a schizophrenic disorder; a disease or condition associated with abnormal plasma hormone levels such as an endocrine disorder; alcohol or drug addiction, symptoms of drug withdrawal or symptoms of alcohol withdrawal; comprising administering a therapeutically effective amount of eszopiclone or a salt thereof, optionally in a pharmaceutical composition, to a subject in need thereof. According to the fifth aspect of the present application, there is also provided a method of improving sleep quality or time, comprising administering an effective amount of eszopiclone or a salt thereof, optionally in a pharmaceutical composition, to a subject in need thereof. Preferably the subject is a mammal, more preferably a human.

### DETAILED DESCRIPTION OF THE INVENTION

The present inventors have surprisingly found that resolution of zopiclone can be made in commercial quantities using a multi-column continuous process or a simulated moving bed process.

The inventors of the present invention have used an amylose derivative of tris(3,5-dimethylphenyl carbamate), a cellulose derivative of tris(3,5-dimethylphenyl carbamate), or an amylose derivative of tris-α-methylbenzylcarbamate as a chiral stationary phase.

The present invention is advantageous in terms of reduction in total number of steps for separation of enantiomers, this being a single step separation. There is no derivatisation being carried out and thus no degradation of zopiclone occurs. The separated optically active enantiomer is pure; therefore no purification step is required.

The present inventors have addressed the need for a multi-column continuous process or a simulated moving bed process for the resolution of eszopiclone, which overcomes all the disadvantages of the resolution processes disclosed in the prior art. Optically pure eszopiclone is resolved from racemic mixture of zopiclone using chiral chromatography.

Simulated moving bed is a method in process chromatography that enables substance mixtures to be continuously separated and extracted in two fractions. By repeated use of the SMB process each partial fraction can be separated into a further fraction—up to binary substance mixtures. Typically, the SMB process is set up in advance for a two-component mixture. Following this, both substances can be immediately extracted in pure form.

Advantages of SMB technology compared to classical batch chromatography:
The entire stationary phase is continuously covered with the mixture to be separated, which produces a much higher productivity.
A 90% reduction in the demand for solvent due to solvent recycling.
High plate counts or particle sizes are no longer required, reducing packing material by 80%.
Extract and raffinate are extracted (in high concentrations) which makes it easier to remove solvent.

A multi-column continuous process or a simulated moving bed process is used for the separation of eszopiclone, which comprises a chiral stationary phase and mobile phase.

The chiral stationary phase used is selected from an amylose derivative of tris(3,5-dimethylphenyl carbamate), a cellulose derivative of tris(3,5-dimethylphenyl carbamate), or an amylose derivative of tris-α-methylbenzylcarbamate.

The mobile phase used is selected from alcohols, organic solvents or mixtures thereof. Alcohol for the mobile phase is selected from methanol, ethanol, propanol, isopropanol or mixtures thereof. Organic solvents are selected from acetonitrile and hexane. Organic amine co-solvents such as dimethylamine can be used in the mobile phase. Mixtures of alcohol and organic solvents can be used as mobile phase. A mixture of acetonitrile and methanol can be used or a mixture of hexane and isopropanol can be used as a mobile phase.

The temperature employed for the present process is at 15-40° C.

The following paragraphs enumerated consecutively from 1 through 37 provide for various aspects of the present invention. In one embodiment, in a first paragraph (1), the present invention provides:
1. A process for optically resolving eszopiclone, comprising chiral chromatography.
2. A process as described in paragraph 1, comprising a multi-column continuous process.
3. A process as described in paragraph 1, comprising a simulated moving bed process.
4. A process as described in any one of the preceding paragraphs, wherein the stationary phase used in the chiral chromatography process comprises an amylose derivative of tris(3,5-dimethylphenyl carbamate), a cellulose derivative of tris(3,5-dimethylphenyl carbamate), or an amylose derivative of tris-α-methylbenzylcarbamate.
5. A process as described in any one of the preceding paragraphs, wherein the stationary phase used in the chiral chromatography process comprises a silica gel coated with a functionalized polysaccharide.
6. A process as described in paragraph 5, wherein the stationary phase comprises Chiralcel® OD, Chiralpak® AD or Chiralpak® AS.
7. A process as described in any one of the preceding paragraphs, wherein the mobile phase used in the chiral chromatography process comprises an alcohol, another organic solvent, or a mixture thereof.
8. A process as described in paragraph 7, wherein the alcohol comprises methanol, ethanol, propanol, isopropanol, or a mixture thereof.
9. A process as described in paragraph 7 or 8, wherein the organic solvent comprises acetonitrile or hexane.
10. A process as described in any one of paragraphs 7 to 9, wherein the mobile phase further comprises an organic amine co-solvent.
11. A process as described in paragraph 10, wherein the organic amine co-solvent is dimethylamine, trimethylamine or isopropylamine.
12. A process as described in any one of paragraphs 7 to 11, wherein the mobile phase comprises a mixture of an alcohol and another organic solvent.
13. A process as described in paragraph 12, wherein the mobile phase comprises a mixture of methanol and acetonitrile.
14. A process as described in paragraph 12, wherein the mobile phase comprises a mixture of isopropanol and hexane.
15. A process as described in any one of the preceding paragraphs, wherein the mobile phase used in the chiral chromatography process is recycled.
16. A process as described in any one of the preceding paragraphs, wherein the chiral chromatography process is carried out at a temperature of 15-40° C.
17. A process as described in any one of the preceding paragraphs, wherein racemic or enantiomerically enriched zopiclone is resolved by chiral chromatography.
18. A process as described in paragraph 17, wherein racemic zopiclone is resolved by chiral chromatography.
19. A process as described in any one of the preceding paragraphs, wherein the process is carried out on an industrial scale.
20. A process as described in paragraph 19, wherein 0.5 kg or more of eszopiclone is produced per day.
21. A process as described in paragraph 20, wherein 1 kg or more of eszopiclone is produced per day.
22. A process as described in paragraph 21, wherein 10 kg or more of eszopiclone is produced per day.
23. A process as described in any one of the preceding paragraphs, wherein the yield of the eszopiclone produced is 70% or more of the theoretical yield.
24. A process as described in paragraph 23, wherein the yield of the eszopiclone produced is 80% or more of the theoretical yield.
25. A process as described in paragraph 24, wherein the yield of the eszopiclone produced is 90% or more of the theoretical yield.
26. A process as described in paragraph 25, wherein the yield of the eszopiclone produced is 95% or more of the theoretical yield.
27. Eszopiclone, or a pharmaceutically acceptable salt thereof, obtained by a process as described in any one of the preceding paragraphs.
28. Eszopiclone or a salt thereof as described in paragraph 27, for use as a medicament.
29. Eszopiclone or a salt thereof as described in paragraph 28, wherein the medicament is suitable for the treatment of anxiety such as acute anxiety, chronic anxiety or a general anxiety disorder; a convulsive state or disorder such as epilepsy or epileptic seizures; an affective disorder such as depression, attention deficit disorder (ADD) or attention deficit disorder with hyperactivity (ADDH); a sleep disorder such as insomnia including situational, transient and chronic insomnia of a primary and secondary nature; aggressive behavior; spasticity or acute muscle spasm; muscle tension; a behavioral disorder; a schizophrenic disorder; a disease or condition associated with abnormal plasma hormone levels such as an endocrine disorder; alcohol or drug addiction, symptoms of drug withdrawal or symptoms of alcohol withdrawal.
30. Eszopiclone or a salt thereof as described in paragraph 28, wherein the medicament is suitable for improving sleep quality or time.
31. A pharmaceutical composition comprising eszopiclone or a salt thereof as described in any one of paragraphs 27 to 30, and a pharmaceutically acceptable carrier or diluent.
32. A pharmaceutical composition as described in paragraph 31, suitable for the treatment of anxiety such as acute anxiety, chronic anxiety or a general anxiety disorder; a convulsive state or disorder such as epilepsy or epileptic seizures; an affective disorder such as depression, attention deficit disorder (ADD) or attention deficit disorder with hyperactivity (ADDH); a sleep disorder such as insomnia including situational, transient and chronic insomnia of a primary and secondary nature; aggressive behavior; spasticity or acute muscle spasm; muscle tension; a behavioral disorder; a schizophrenic disorder; a disease or condition associated with abnormal plasma hormone levels such as an endocrine disorder; alcohol or drug addiction, symptoms of drug withdrawal or symptoms of alcohol withdrawal.
33. A pharmaceutical composition as described in paragraph 31, suitable for improving sleep quality or time.
34. Use of eszopiclone or a salt thereof as described in any one of paragraphs 27 to 30, for the manufacture of a medicament for the treatment of anxiety such as acute anxiety, chronic anxiety or a general anxiety disorder; a convulsive state or disorder such as epilepsy or epileptic seizures; an affective disorder such as depression, attention deficit disorder (ADD) or attention deficit disorder with hyperactivity (ADDH); a sleep disorder such as insomnia including situational, transient and chronic insomnia of a primary and secondary nature; aggressive behavior; spasticity or acute muscle spasm; muscle tension; a behavioral disorder; a schizophrenic disorder; a disease or condition associated with abnormal plasma hormone levels such as an endocrine disorder; alcohol or drug addiction, symptoms of drug withdrawal or symptoms of alcohol withdrawal.
35. Use of eszopiclone or a salt thereof as described in any one of paragraphs 27 to 30, for the manufacture of a medicament for improving sleep quality or time.
36. A method of treating anxiety such as acute anxiety, chronic anxiety or a general anxiety disorder; a convulsive state or disorder such as epilepsy or epileptic seizures; an affective disorder such as depression, attention deficit disorder (ADD) or attention deficit disorder with hyperactivity (ADDH); a sleep disorder such as insomnia including situational, transient and chronic insomnia of a primary and secondary nature; aggressive behavior; spasticity or acute muscle spasm; muscle tension; a behavioral disorder; a schizophrenic disorder; a disease or condition associated with abnormal plasma hormone levels such as an endocrine disorder; alcohol or drug addiction, symptoms of drug withdrawal or symptoms of alcohol withdrawal; comprising administering a therapeutically effective amount of eszopiclone or a salt thereof as described in any one of paragraphs 27 to 30, or a pharmaceutical composition as described in any one of paragraphs 31 to 33, to a subject in need thereof.
37. A method of improving sleep quality or time, comprising administering an effective amount of eszopiclone or a salt thereof as described in any one of paragraphs 27 to 30, or a pharmaceutical composition as described in any one of paragraphs 31 to 33, to a subject in need thereof.

While the present invention has been described in terms of its specific embodiments, certain modifications and equivalents will be apparent to those skilled in the art and are intended to be included within the scope of the present invention. The details of the invention, its objects and advantages are explained hereunder in greater detail in relation to non-limiting exemplary illustrations.

### EXAMPLE

Racemic zopiclone was subjected to preparative chromatography using Chiralcel® OD as the stationery phase and isopropanol as the mobile phase. Under these conditions the crude material has a good solubility in the mobile phase (>1.0 g/liter) and the typical retention time for the R-isomer is 12.3 minutes and for the S-isomer is 17.8 minutes. Some physico-chemical properties of the S-zopiclone obtained are set out in table 1.

As single solvent is used, it can be recycled with a minor loss of <0.1% on an industrial scale.

TABLE 1
Physico-chemical properties of S-Zopiclone

| Tests | S-Zopiclone |
|---|---|
| Appearance | off-white powder |
| Enantiomeric purity | 98.98% |
| (by HPLC) | |
| Specific optical | +134.2° |
| rotation | |
| (1% in acetone at | |
| 25° C.) | |
| UV (methanol) | 211.4 and 305.1 |
| λₘₐₓ nm | |
| ESI-MS (M + H)⁺ | m/z 389.3 (58%), 391.3 (31%) |
| ¹H NMR (300 MHz, | 8.87 (dd, J = 12.8, 2.4 Hz, 2H), 8.52 (d, J = 8.9 Hz, |
| CDCl₃, δ) | 1H), 8.40 (d, J = 1.5 Hz, 1H), 8.02 (s, 1H), |
| | 7.80 (dd, J = 8.9, 2.5 Hz, 1H), 3.65 (broad, 1H), |
| | 3.55 (broad, 1H), 3.25 (bs, 2H), 2.41 (broad, |
| | 2H), 2.25 (s, 3H), 2.20 (broad, 1H) and 2.03 (m, |
| | 1H) |

It will be understood that the present invention has been described above by way of example only. The example is not intended to limit the scope of the invention. Various modifications and embodiments can be made without departing from the scope and spirit of the invention, which is defined by the following claims only.

What is claimed is:

1. A chiral chromatographic process for optically resolving eszopiclone, comprising contacting eszopiclone with a chiral chromatographic stationary phase and a mobile phase.

2. A process as claimed in claim 1, comprising a multi-column continuous process or a simulated moving bed process.

3. A process as claimed in claim 1, wherein the stationary phase used in the chiral chromatography process comprises:
(a) an amylose derivative of tris(3,5-dimethylphenyl carbamate), a cellulose derivative of tris(3,5-dimethylphenyl carbamate), or an amylose derivative of tris-α-methylbenzylcarbamate; and/or
(b) a silica gel coated with a functionalized polysaccharide; and/or
(c) Chiralcel® OD, Chiralpak® AD or Chiralpak® AS.

4. A process as claimed in claim 1, wherein the mobile phase used in the chiral chromatography process comprises:
(a) an alcohol, another organic solvent, or a mixture thereof; and/or
(b) methanol, ethanol, propanol, isopropanol, or a mixture thereof; and/or
(c) acetonitrile, hexane, or a mixture thereof; and/or
(d) a mixture of an alcohol and another organic solvent; and/or
(e) a mixture of methanol and acetonitrile; and/or
(f) a mixture of isopropanol and hexane.

5. A process as claimed in claim 1, wherein the mobile phase further comprises an organic amine co-solvent.

6. A process as claimed in claim 5, wherein the organic amine co-solvent is dimethylamine, trimethylamine or isopropylamine.

7. A process as claimed in claim 1, wherein the mobile phase used in the chiral chromatographic process is recycled.

8. A process as claimed in claim 1, wherein the chiral chromatographic process is carried out at a temperature of 15-40° C.

9. A process as claimed in claim 1, wherein racemic or enantiomerically enriched zopiclone is resolved by chiral chromatography.

10. A process as claimed in claim 1, wherein the process is carried out on an industrial scale.

11. A process as claimed in claim 10, wherein 0.5 kg, or 1 kg, or 10 kg, or more of eszopiclone is produced per day.

12. A process as claimed in claim 1, wherein the yield of the eszopiclone produced is 70%, or 80%, or 90%, or 95%, or more of the theoretical yield.

13. Eszopiclone, or a pharmaceutically acceptable salt thereof, obtained by a process as claimed in claim 1.

14. A pharmaceutical composition comprising eszopiclone or a salt thereof as claimed in claim 13, and a pharmaceutically acceptable carrier or diluent.

15. A method of treating anxiety such as acute anxiety, chronic anxiety or a general anxiety disorder; a convulsive state or disorder such as epilepsy or epileptic seizures; an affective disorder such as depression, attention deficit disorder (ADD) or attention deficit disorder with hyperactivity (ADDH); a sleep disorder such as insomnia including situational, transient and chronic insomnia of a primary and secondary nature; aggressive behavior; spasticity or acute muscle spasm; muscle tension; a behavioral disorder; a schizophrenic disorder; a disease or condition associated with abnormal plasma hormone levels such as an endocrine disorder; alcohol or drug addiction, symptoms of drug withdrawal or symptoms of alcohol withdrawal; comprising administering a therapeutically effective amount of eszopiclone or a salt thereof as claimed in claim 13, to treat a subject in need thereof.

16. A method of improving sleep quality or time, comprising administering a therapeutically effective amount of eszopiclone or a salt thereof as claimed in 13, to treat a subject in need thereof.

17. A method of treating anxiety such as acute anxiety, chronic anxiety or a general anxiety disorder; a convulsive state or disorder such as epilepsy or epileptic seizures; an affective disorder such as depression, attention deficit disorder (ADD) or attention deficit disorder with hyperactivity (ADDH); a sleep disorder such as insomnia including situational, transient and chronic insomnia of a primary and secondary nature; aggressive behavior; spasticity or acute muscle spasm; muscle tension; a behavioral disorder; a schizophrenic disorder; a disease or condition associated with abnormal plasma hormone levels such as an endocrine disorder; alcohol or drug addiction, symptoms of drug withdrawal or symptoms of alcohol withdrawal; comprising administering a therapeutically effective amount of a pharmaceutical composition as claimed in claim 14, to treat a subject in need thereof.

18. A method of improving sleep quality or time, comprising administering a therapeutically effective amount of a pharmaceutical composition as claimed in claim 14, to treat a subject in need thereof.
